(19) United States
(12) Patent Application Publication
(10) Pub. No.: US 2024/0157144 A1
(43) Pub. Date: May 16, 2024

(54) CLOSED-LOOP DEEP BRAIN STIMULATION USING NEURAL AND BEHAVIORAL BIOMARKERS OF SPECIFIC MOTOR FEATURES

(71) Applicants: Brown University, Providence, RI (US); Rhode Island Hospital, Providence, RI (US)

(72) Inventors: Peter Lauro, Providence, RI (US); Shane Lee, Providence, RI (US); Umer Akbar, Providence, RI (US); Wael Asaad, Providence, RI (US)

(21) Appl. No.: 18/388,759

(22) Filed: Nov. 10, 2023

Related U.S. Application Data
(60) Provisional application No. 63/424,322, filed on Nov. 10, 2022.

(51) Int. Cl. A61N 1/36; A61B 5/00; A61B 5/11; A61B 5/388

(52) U.S. Cl. CPC A61N 1/36067; A61B 5/1101; A61B 5/388; A61B 5/4836; A61B 5/7267; A61N 1/36139; A61N 1/36153; A61N 1/0534

(57) ABSTRACT

Closed-loop deep brain stimulation using neural and behavioral biomarkers of specific motor features is provided via training a machine learning model to differentiate first neural signals generated by a biological subject that are associated with tremor in the biological subject from second neural signals generated by the biological subject that are associated with bradykinesia in the biological subject; placing a first plurality and a second plurality of deep brain stimulation electrodes in a subthalamic nucleus (STN) of the biological subject; placing a plurality of microelectrodes and a plurality of macroelectrodes in the STN; in response to identifying, by the machine learning model, a given neural signal as one of the first or second neural signals, activating a corresponding one of the first or second plurality of electrodes with a therapeutically effective voltage and current to affect deep brain stimulation in the biological subject to treat a neuromotor disorder.

### CROSS REFERENCES TO RELATED APPLICATIONS

The present disclosure claims the benefit of U.S. Provisional Patent Application No. 63/424,322 entitled “CLOSED-LOOP DEEP BRAIN STIMULATION USING NEURAL AND BEHAVIORAL BIOMARKERS OF SPECIFIC MOTOR FEATURES” and filed on Nov. 10, 2022, which is incorporated herein by reference in its entirety.

### TECHNICAL FIELD

The present disclosure relates to tools for managing Deep Brain Stimulation for treating Parkinson's disease and other neurological diseases.

### SUMMARY

The present disclosure provides new and innovative systems and methods for the use of closed-loop deep brain stimulation using neural and behavioral biomarkers of specific motor features in the treatment of Parkinson's disease and other neuromotor diseases.

Extant approaches to closed-loop deep brain stimulation rely on generic, nonspecific biomarkers to drive stimulation. However, diseases such as Parkinson's disease are characterized by different forms of motor dysfunction, and these dimensions of motor pathology are characterized by different and even opposing neurophysiological and behavioral features. Therefore, the methods and systems described herein allow an improved therapeutic strategy to address each aspect of motor and non-motor dysfunction. The described methods and systems use multiple control signals to drive the location, type, and timing of stimulation in order to address distinct aspects of disease expression.

In various aspects, a method, a system for performing the method, and various goods produced by the method are provided. In various aspects, the method includes: training a machine learning model to differentiate first neural signals generated by a biological subject that are associated with tremor in the biological subject from second neural signals generated by the biological subject that are associated with bradykinesia in the biological subject; placing a first plurality of deep brain stimulation (DBS) electrodes in a subthalamic nucleus (STN) of the biological subject; placing a second plurality of DBS electrodes in the STN of the biological subject; placing a plurality of microelectrodes in the STN of the biological subject; placing a plurality of macroelectrodes in the STN of the biological subject; and in response to identifying, by the machine learning model, a given neural signal observed by the plurality of microelectrodes and the plurality of macroelectrodes as one of the first neural signals or the second neural signals, activating a corresponding one of the first plurality of DBS electrodes or the second plurality of DBS electrodes with a therapeutically effective voltage and current to affect deep brain stimulation in the biological subject to treat a neurological disorder in the biological subject.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

FIG. 1 illustrates an example setup for performing deep brain stimulation, according to embodiments of the present disclosure.

FIGS. 2A and 2B illustrate views of cursor tracing and processing for use in performing deep brain stimulation, according to embodiments of the present disclosure.

FIGS. 3A-3J illustrates plotted data from cursor tracing and processing for use in performing deep brain stimulation, according to embodiments of the present disclosure.

FIGS. 4A-4C illustrate plotted data for different motor effects discernable, according to embodiments of the present disclosure.

FIG. 5 is a flowchart for an example method of closed-loop deep brain stimulation using neural and behavioral biomarkers of specific motor features, according to embodiments of the present disclosure.

FIG. 6 illustrates a computing device, according to embodiments of the present disclosure.

### DETAILED DESCRIPTION

The present disclosure provides new and innovative systems and methods for the use of closed-loop deep brain stimulation using neural and behavioral biomarkers of specific motor features in the treatment of Parkinson's disease and other neurological diseases.

Extant approaches to closed-loop deep brain stimulation rely on generic, nonspecific biomarkers to drive stimulation. However, diseases such as Parkinson's disease are characterized by different forms of motor dysfunction and these dimensions of motor pathology are characterized by different and even opposing neurophysiological and behavioral features. Therefore, the methods and systems described herein allow an improved therapeutic strategy to address each aspect of motor dysfunction. The described methods and systems use multiple control signals to drive the location, type and timing of stimulation in order to address distinct aspects of disease expression.

Motor signs of Parkinson's disease (and other neuromotor diseases) such as tremor and bradykinesia can be independently decoded from subthalamic and cortical recordings, and may be used to improve treatment for patients.

Parkinson's Disease (PD) is characterized by distinct motor phenomena that are expressed asynchronously. Understanding the neurophysiological correlates of these different motor states can facilitate monitoring of disease progression and allow for improved assessments of therapeutic efficacy, as well as enable optimal closed-loop neuromodulation. The system analyzes neural activity in the basal ganglia and cortex of subjects with PD during a quantitative motor task to decode tremor and bradykinesia—two cardinal motor signs of this disease—and relatively asymptomatic periods of behavior. Support-vector regression analysis of subcortical microelectrode and cortical electrocorticography recordings revealed that tremor and bradykinesia have distinct, nearly opposite neural signatures, while effective motor control displayed unique, differentiating features. The neurophysiological signatures of these motor states depended on the type of signal recorded as well as the location. Overall, cortical decoding accuracy generally outperformed subcortical decoding; however, within the subthalamic nucleus (STN), while tremor and bradykinesia were better decoded from different distinct subregions portions of the subthalamic nucleus (STN). These results provide a roadmap to leverage real-time neurophysiology to understand and treat PD.

Parkinson's disease (PD) is a common and complex neurodegenerative disorder characterized by the dynamic expression of particular motor features such as tremor and bradykinesia. These distinct motor signs are expressed variably across patients and may respond differently to dopamine replacement therapy; the differential expression of these motor signs often is used to classify patients into phenotypic subtypes. Despite this heterogeneity, both of these motor features (and tremor-dominant and non-tremor-dominant patient subtypes) respond to high-frequency deep brain stimulation (DBS) applied to the subthalamic nucleus (STN).

DBS delivered in a closed-loop fashion (i.e., in response to neurophysiological biomarkers) has shown promising therapeutic potential primarily toward alleviating bradykinesia, but current efforts focusing on β-frequency oscillations (15-30 Hertz (Hz)) have been shown to inadequately treat or even worsen tremor in some cases. Thus, tremor may be better signaled by different components within the local field potential (LFP) spectrum, and closed-loop DBS strategies could benefit from a clearer understanding of the neurophysiological biomarkers that differentiate these motor signs from each other, and from more optimal motor performance in the absence of these impairments.

To this point, STN LFP recordings from patients with different PD subtypes have revealed distinct patterns of oscillatory activity. In addition to spectral variability, specific stimulation sites within the STN have been associated with the preferential reduction of individual motor signs. Moreover, these STN sites were associated with specific patterns of anatomical connectivity with cortical structures. Much like how overlapping subdivisions of basal-ganglia-cortical circuits have been found to encode separate aspects of movement, separate motor features may be mediated by different sub-circuits involving the STN and sensorimotor cortex.

In order to better reveal the functional and anatomical substrates of distinct PD motor states, patients with PD undergoing awake DBS electrode implantation were observed to perform a continuous visual-motor task that allowed rigorous, concurrent measurement of different motor metrics while acquiring STN (micro- and macro-electrode) and cortical (electrocorticography; ECoG) recordings. Prior studies have not attempted to simultaneously decode different aspects of disease expression, contrast these measures with symptom-free performance, and examine disease expression on the short timescales relevant to that varying expression. Although the ability to decode global PD motor dysfunction from STN recordings on short timescales has previously been shown, the focus of the present disclosure on individual motor features and the specific neurophysiological manifestations thereof are used to train machine learning models to directly decode tremor or slowness from neural recordings to reveal the spectral and anatomical fingerprints of these cardinal motor features of PD.

With advances in technology, DBS aspires towards incorporating chronic neurophysiological recordings to help guide therapeutic stimulation. While currently-applied closed-loop DBS paradigms trigger stimulation based on one or two frequency bands representing PD symptoms, the present model provides for utility of a more fine-grained and targeted neurophysiological approach to PD state identification: more dorsal STN contacts may better sense signals reflecting tremor, while more ventral STN contacts may better identify signals corresponding to bradykinesia. Precise symptom-specific models could not only inform where to stimulate but also when and how to stimulate (i.e., identifying stimulation settings to best treat tremor vs. bradykinesia). Most importantly, future neuromodulation paradigms could be derived not simply to disrupt pathological activity but actually to sustain the neurophysiological γ frequency “targets” of effective motor control. Looking ahead, chronic cortical recordings could work in concert with STN recordings to help identify precise motor states associated with specific aspects of disease expression.

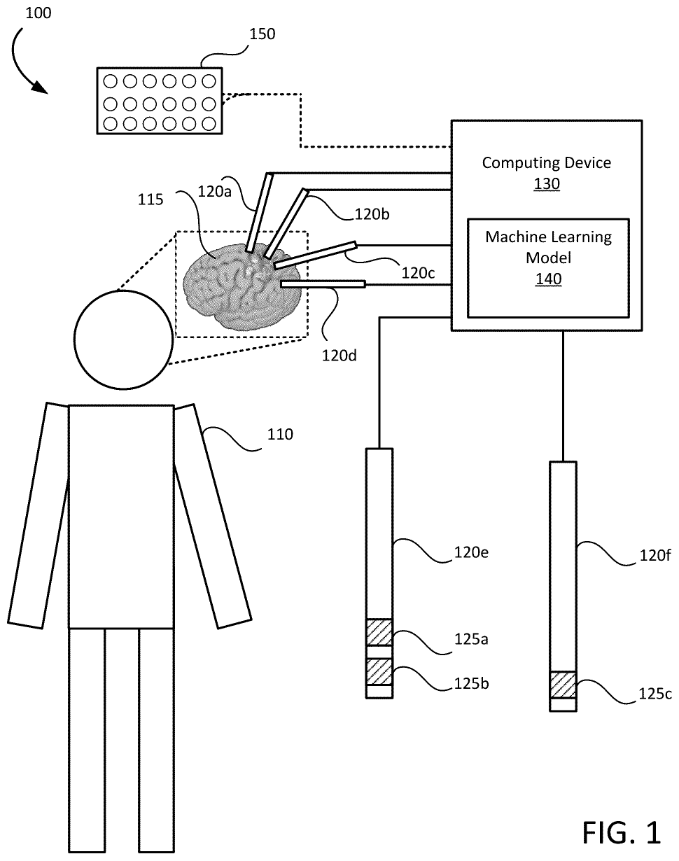

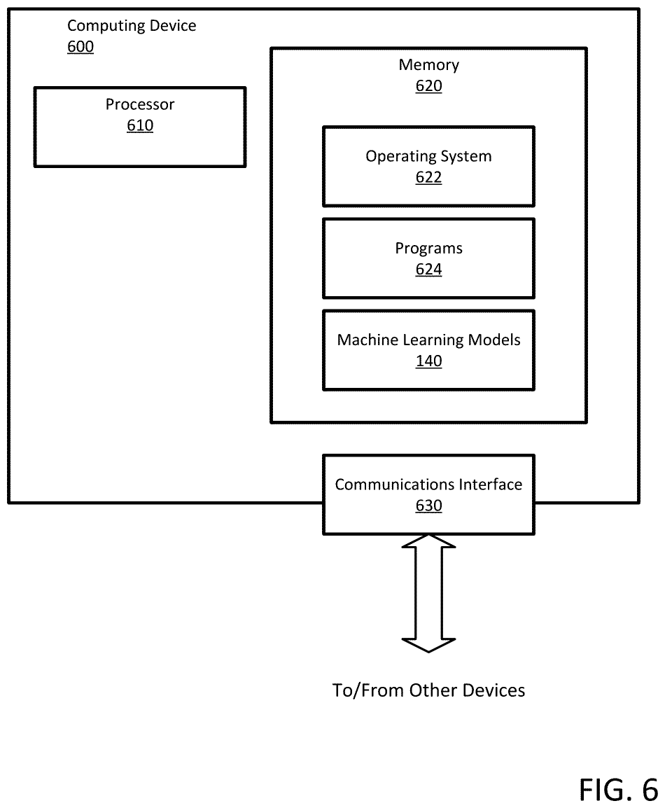

FIG. 1 illustrates an example setup 100 for performing deep brain stimulation, according to embodiments of the present disclosure. As shown, a plurality of electrodes 120a-f (generally or collectively, electrodes 120) are inserted into the brain 115 of a biological subject 110. The electrodes 120 are communicated to a computing device 130 (such as the computing device 600, described in greater detail in regard to FIG. 6) that provides a machine learning model 140 to interpret the signals received from the biological subject 110 and determine whether to send back DBS signals to one or more of the electrodes 120 for the treatment or prophylaxis of a neuromotor disorder originating in the brain 115.

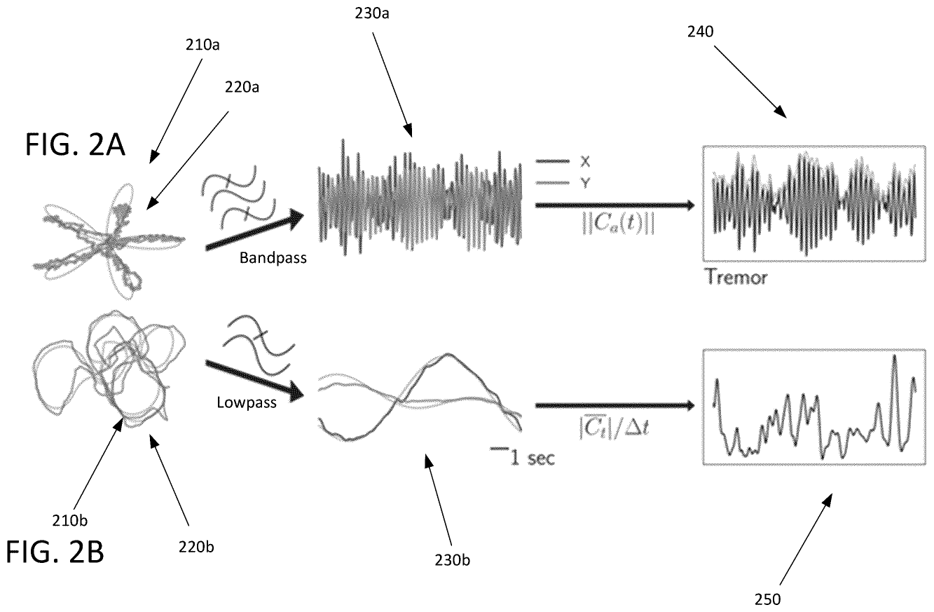

In various embodiment, the electrodes 120 are placed in the subthalamic nucleus of the brain 115. Microelectrode recordings (MER) from the region of the STN of awake patients are routinely obtained in order to map the target area and guide DBS electrode implantation. Microdrives attached to a patient-customized stereotactic platform may be loaded with three parallel microelectrodes. Additionally or alternatively, electrocorticography (ECoG) strips 150 can be placed posteriorly along sensorimotor cortices through the same burr hole used for MER insertion to conduct intraoperative cortical recordings. The STN was identified electrophysiologically as a hyperactive region typically first encountered about 3-6 millimeters (mm) above estimated target. When at least one electrode 120 was judged to be within the STN, electrode movement was paused and recordings were obtained in conjunction with patient performance of a visual-motor task (e.g., as shown in FIGS. 2A-2B). In some embodiments, once the bottom of the STN was identified using typical electrophysiological procedures, custom-built routines using an FDA-approved software development kit are used to automatically raise the electrodes 120 by a pre-specified distance between trials to conduct a high-density STN survey.

In various embodiments, the biological subject 110 may be a human or other animal that is subject to a neuromotor disorder (such as PD), is suspected of being subject to a neuromotor disorder, or is not subject to such a disorder (e.g., as a control or training aid for the machine learning model 140). The electrodes 120 may be placed for purposes of training the machine learning model 140, treatment or prophylaxis of a neuromotor disorder, or both.

The various electrodes 120 are surgically implanted at various locations along anatomical trajectories for neural signals along the brain of the biological subject, as will be familiar to the skilled practitioner. In various embodiments, a single electrode 120 may include one or more contacts 125a-c (generally or collectively contacts 125) by which the electrical activity of neural signals are measured or by which a voltage and current are applied to affect deep brain stimulation in the biological subject. In various examples, separate first and second pluralities of electrodes 120 are used for DBS in different regions of the brain based on measurements collected by a third plurality of electrodes in a third region of the brain. In various embodiments, the measurement electrodes include two contacts 125a-b for use as macroelectrodes and microelectrodes (with located the macroelectrode contact 125a positioned superior, relative to the biological subject 110, to the microelectrode contact 125b). In various embodiments, the pluralities of DBS electrodes 120 are provided with a single contact 125c or provide the same voltage and current via two contacts 125a-b (e.g., acting as a single contact 125 over a distributed area).

### Example 1

In one example of the experimental setup shown in FIG. 1, a total of 203 microelectrode and 176 macroelectrode recordings (microelectrode tips and macroelectrode contacts separated by 3 mm on the same electrodes 120) were acquired from the STN as subjects performed a motor task of tracing, via a computer cursor, a pattern displayed on a screen. To assess whether tremor or slowness could be decoded from these recordings, spectral estimates of power from 3-400 Hz were obtained using a wavelet convolution. Narrowband power estimates were grouped into six broad frequency bands (θ/θ/α,β, γ_{low}, γ_{mid}, γ_{high}, and high-frequency oscillations (hfo)) with seven sub-bands each, for a total of 42 neural “features” per 7-second epoch. Neural decoding models (e.g., support vector regression (SVR) or SVR with a linear kernel) were trained directly on the epoch's average metric (e.g., tremor or slowness values averaged within each epoch), and the performance of the models was assessed with Pearson's r between observed and decoded metrics. Across all microelectrode recordings, tremor decoding performance (r=0.196±0.250) was superior to slowness decoding (r=0.114±0.193) (n=203 microelectrode recording models, tremor v. slowness, LMM β=0.083, Z=4.626, p=4.00*10−6). No such difference was observed across macroelectrode recordings (n=176 macroelectrode recordings models, tremor v. slowness, r=0.208±0.283 v. r=0.217±0.274, LMM β=−0.009, Z=−0.557, p=0.578).

To determine whether tremor and slowness had distinct neurophysiological signatures, SVR model feature weights were aggregated for each metric. To understand which spectral features were used consistently across models, feature weights were compared to null distributions generated from models where motor metric values were shuffled with respect to the corresponding spectral features. Microelectrode tremor decoding models positively weighted low-frequency features (θ,α,β, 4-21 Hz; p<0.001, permutation test. Hfo (275-375 Hz) weights were also positively associated with tremor decoding (p<0.014, permutation test). In contrast, macroelectrode tremor decoding models negatively weighed β power (14-41 Hz; p<0.026, permutation test) while positively weighing γ/hfo activity (60-375 Hz, p<0.011, permutation test). In other words, optimal macroelectrode tremor decoding relied on decreased β power and increased γ/hfo power.

For slowness, microelectrode decoding models had negative θ, γ_{low}, and hfo weights (5-12 Hz; 33-56 Hz; 200-375 Hz) (p<0.012, permutation test). Macroelectrode decoding models positively weighted β frequencies (12-30 Hz; p<0.006, permutation test) along with negative γ/hfo weights (33-375 Hz; p<0.001, permutation test). Tremor and slowness model features differed when compared directly, with hfo frequencies (225-375 Hz) being elevated during tremor in both micro-/macro-electrode recordings. Overall, just as tremor and slowness represented two distinct, anti-correlated symptomatic states of PD motor dysfunction, tremor and slowness decoding models from the STN revealed distinguishable patterns of underlying neural activity.

However, in order to rule out the possibility that the alternating patterns of relevant neural decoding features simply reflected the anti-correlated nature of tremor and slowness, the decoding models trained for tremor were tested to determine whether those models could also accurately decode slowness. When directly comparing tremor and slowness decoding performance on tremor-trained models, slowness decoding was inferior for both microelectrode (tremor v. slowness decoding, r=0.194±0.253 v. 0.000±0.025; LMM β=0.195, Z=12.373 p=3.67*10−35) and macroelectrode (tremor v. slowness decoding, r=0.205±0.213 v. −0.005±0.024; LMM β=0.210, Z=14.360, p=9.28*10−47) recordings. If decoding features for tremor and slowness were simply inverted, applying models for decoding another metric would result in negative r-values above chance. Thus, the neural features used for individual metric decoding likely reflected a unique spectral state or “fingerprint.”

From these tests, it was determined that effective motor control had characteristic neural signatures. For example, PD produces a fluctuating motor deficit such that there can be moments of normal-appearing motor behavior. Accordingly, there may be a neural activity that represented these “effective” motor states. Specifically, epochs with lower tremor and/or higher movement speeds were assigned values closer to 1 while more symptomatic epochs (e.g., high tremor and/or slower movement speeds) were assigned values closer to 0. Compared to other metrics, effective motor control was expressed on longer timescales (FWHM=3.784 s, median state length=7.900 s)

Effective motor control was similarly decoded from both micro-(r=0.138±0.194) and macro-electrode (r=0.228±0.181) recordings. Effective motor control decoding was characterized by the absence of β (10-28 Hz) power in both microelectrode and macroelectrode recordings (p<0.006, permutation test), while macroelectrodes also exhibited positive γ power weights (30-175 Hz; p<0.020, permutation test). Power in γ_{low}, frequencies (30-48 Hz) in particular was significantly increased during effective motor control relative to both tremor and slowness decoding models (p<0.006, permutation test). In total, STN activity contained specific features that distinguished symptomatic from non-symptomatic motor states. Tremor was characterized by lower frequencies (θ/α) in microelectrodes, slowness by β frequencies in macroelectrodes, and effective motor performance was uniquely characterized by γ_low frequencies from both recording types.

To directly test whether each behavior model used neural features across the spectrum, the relative abilities of full-spectrum and canonical band (β, 12-30 Hz) models were assessed. Full-spectrum decoding had significantly greater performance for microelectrode (full vs. beta-only decoding, LMM β=0.034-0.045, Z=2.036-2.994, p<0.042) and macroelectrode (full vs. beta-only decoding, LMM β=0.041-0.063, Z=2.561-3.745, p<0.010) for all three metrics. ECoG recordings full-spectrum decoding was superior for tremor and effective motor control (full vs. beta-only decoding, LMM β=0.053-0.058, Z=3.139-3.512, p<0.002), but equivocal for slowness (full vs. beta-only decoding, LMM β=0.008, Z=0.522, p=0.602).

It was determined that optimal subthalamic tremor decoding sites were dorsolateral to optimal slowness-decoding sites across subjects, To investigate whether tremor and slowness were more optimally decoded from distinct areas within the STN, recording sites for each session were reconstructed using subject-specific neuroimaging (peak microelectrode recording density in MNI space: x=−12, y=−10, z=−6.0); peak macroelectrode recording density: x=−12, y=−9, z=−3.0). For each recording site, the corresponding decoding model performance for each metric was plotted.

The voxel-wise relative performance was compared between tremor and slowness throughout all recorded STN voxels by using a modified 3D t-test with spatially-based permutation shuffling. Tremor was better decoded in recordings from dorsolateral STN (n=182 microelectrode recording sites, x=−14.0, y=−13.0, z=−5.0, Z=2.207, p=0.014), whereas slowness was better decoded from recordings in central/ventromedial STN (x=−11.0, y=−13.0, z=−8.0, Z=1.915, p=0.028).

Optimal locations for tremor and slowness decoding were not found to differ significantly by macroelectrode location (n=176 macroelectrode recording sites, p>0.05). Moreover, the locus of optimal effective motor control decoding was not observed to differ from those of tremor or speed using either micro- and macro-electrode recordings (p>0.05). Nevertheless, it was found that that differences in metric decoding were not only related to the frequencies present, but also to an electrode's location within the STN, as assessed over the entire study PD population.

From these assessments, it was determined that the optimal subthalamic tremor decoding sites were dorsolateral to optimal slowness decoding sites within individual subjects. To verify the spatial relationship of optimal tremor and slowness decoding within subjects, five additional right-handed patients (70.0±8.9 years old; 2F, 3M; UPDRS III: 45.2±9.5) underwent a modified version of the random-pattern task. Rather than acquire recordings from a stationary site, here the entire length of the STN was observed by systematically moving the electrodes between task trials in small, discrete steps.

SVR models for tremor and slowness were then calculated by incorporating recording data across all sites/trials within a single trajectory. Although decoding performance of models derived from multi-site data exhibited a trend of lower performance than models trained on single-site recordings (Tremor: r=0.196±0.250 v. 0.094±0.141; Slowness: r=0.114±0.193 v. 0.067±0.144, Effective motor control: r=0.138±0.194 v. 0.055±0.141), these differences were not significant (n=203 stationary microelectrode recording sites, n=17 moving microelectrode recording trajectories, moving v. stationary data, LMM β=−0.053-−0.102, Z=−0.766-−1.057, p=0.222-0.444). Despite the lack of data at each recording site, whole-STN models demonstrated above-chance decoding performance for all three metrics (tremor: 8/17 trajectories, slowness: 14/17, effective motor control: 10/17).

Recording sites along each trajectory were also reconstructed using imaging, and site-specific metric decoding r-values were calculated by applying the whole-STN SVR model to individual site recordings. Decoding performance was then plotted across the subjects. Across these recordings, tremor was optimally decoded at x=−13.0, y=−13.0, z=−5.0 (Z=1.817, p=0.035), while slowness was optimally decoded at x=−12.0, y=−13.0, z=−8.0 (Z=2.050, p=0.020). Again, tremor was found to be decoded dorsolaterally to slowness within individual subjects.

Cortical recordings also revealed distinct representations of tremor, slowness, and effective motor control. Ten subjects additionally had ECoG recordings from sensorimotor cortex (motor cortex: n=16 contacts, somatosensory cortex: n=15, see Methods). SVR models for metric decoding were similarly trained on ECoG signals. ECoG decoding performance did not differ between tremor or slowness (n=85 ECoG recordings across 27 sessions and 10 subjects, tremor: r=0.322±0.215, slowness: r=0.307±0.212, tremor v. slowness, LMM β=0.015, Z=0.557, p=0.578).

To understand which spectral features contributed to cortical metric decoding, SVR model weights were aggregated across all patients and recordings and compared to metric-shuffled models. When compared directly, cortical tremor and slowness models had opposing relationships in α/β (8-40 Hz, p<0.027, permutation test), γ_{mid} (45-125 Hz, p<0.023, permutation test), and γ_{high} (150-225 Hz, p<0.015, permutation test) frequency bands. Tremor models additionally had positive weights associated with θ frequencies (5-7 Hz, p=0.003, permutation test). Altogether, although cortical signals supported equivalent decoding performance for tremor or slowness, decoding features were nonetheless distinct.

On the other hand, effective motor control decoding performance (r=0.224±0.177) was lower than tremor (effective motor control v. tremor, LMM β=−0.097, Z=−3.619, p=2.96*10−4) and slowness (effective motor control v. slowness, LMM β=−0.082, Z=−3.062, p=0.002). Nevertheless, effective motor control was represented in cortical decoding models by γ_{high} frequencies. These γ_{high} features additionally appeared to differentiate effective motor control models from both tremor and slowness models (125-175 Hz, p<0.026, permutation test). In addition, α/β (8-30 Hz, p<0.001, permutation test) and γ_{low} (45-75 Hz, p<0.010, permutation test) frequencies exhibited an opposing relationship between effective motor control and slowness, much like the interaction between tremor and slowness models). Taken together, although at different γ frequencies, both STN (γ_{low}) and sensorimotor cortex (γ_{high}) exhibited features specific to effective motor control.

Finally, it was analyzed whether motor features were selectively represented in different regions of cortex. ECoG recording sites (and the associated metric decoding performance thereof) were plotted along a standard cortical surface. Comparing tremor and slowness decoding performance by cortical anatomy revealed that slowness decoding had peaks in medial motor (n=31 ECoG recording sites, x=−37.4, y=−15.8, z=+70.2; T=2.332, p=0.022) and somatosensory (x=−33.6, y=−37.5, z=+71.2, T=2.034, p=0.043) cortices. A trending tremor decoding peak in lateral somatosensory cortex was also observed (x=−46.4, y=−30.9, z=+67.1; T=1.650, p=0.10). Similar to the STN, effective motor control decoding performance (relative to tremor or slowness decoding) was found not to differ by cortical anatomy (p>0.05). In sum, optimal tremor cortical decoding sites were found along relatively more lateral somatosensory cortices, whereas optimal slowness decoding sites were observed to be relatively medial to tremor sites.

To understand whether motor function or dysfunction was better represented in cortical signals, decoding performance was compared between subjects with ECoG and STN recordings (n=10 subjects, 85 ECoG recordings, 81 microelectrode recordings, 81 macroelectrode recordings). ECoG tremor decoding models exhibited higher performance than micro-STN recordings (ECoG v. micro-STN, r=0.322±0.215 v. 0.204±0.236, LMM β=0.109, Z=3.981, p=6.86*10−5), but only trended higher relative to macro-STN recordings (ECoG v. macro-STN, r=0.322±0.215 v. 0.280±0.219, LMM β=0.034, Z=1.232, p=0.218). Slowness decoding performance on the other hand was higher in ECoG models relative to both macro-STN (ECoG v. macro-STN, r=0.307±0.212 v. 0.190±0.205, LMM β=0.114, Z=4.382, p=1.18*10−5) and micro-STN (ECoG v. micro-STN, r=0.307±0.212 v. 0.109±0.175, LMM β=0.195, Z=7.511, p=5.86*10−14) recordings. Like tremor, effective motor control ECoG decoding models exhibited superior decoding performance to micro-STN (ECoG v. micro-STN, r=0.224±0.177 v. 0.111±0.176, LMM β=−0.114, Z=−4.409, p=1.04*10-5), but not macro-STN (ECoG v. macro-STN, r=0.224±0.177 v. 0.206±0.186, LMM β=0.018, Z=0.708, p=0.479) recordings. In summary, recordings from sensorimotor cortex were superior to micro-STN and macro-STN recordings for decoding slowness, while cortical recordings were also superior to micro-STN recordings for decoding tremor and effective motor control.

The data show that the STN exhibited clear functional and anatomical topography. The described neural decoding approach focuses on two cardinal motor features of PD to isolate spectral features that reflected the expression of each. In the STN, tremor was characterized by lower frequency (θ, α) oscillations in microelectrodes, whereas slowness was characterized by the presence of β oscillations and the absence of γ oscillations in macroelectrodes. Because γ frequency oscillations are commonly associated with hyperkinetic states, the slowness decoding results may be understood in part as an “anti-speed” neural model. Indeed, effective motor control was distinguished by γ_{low} frequency activity, highlighting the importance of γ frequency oscillations in effective movements. Some of these frequency bands in isolation (e.g., θ, β) have been found to correlate with clinical measures of tremor and bradykinesia. However, here the data show directly the contrasting nature of distinct PD motor states both behaviorally and neurophysiologically, and highlight the dependence of these neurophysiological “fingerprints” on the particular neural recording technique.

The described methodology also identifies where tremor and slowness are optimally decoded (e.g., where metric-specific spectral information was greatest). Within the STN microelectrode recordings, optimal tremor decoding sites were found to be located within dorsolateral STN, whereas optimal slowness decoding sites were more centrally located within the STN. Considering the potential imprecision of pooling recording sites across multiple subjects, optimal tremor decoding may have included activity from zona incerta (ZI), a stimulation site commonly thought to be critical for alleviating tremor. Indeed, optimal stimulation sites to alleviate tremor and bradykinesia correspond to the dorsolateral-tremor/ventromedial-slowness topography. While several groups have localized β frequency activity to dorsolateral STN, this has been observed to be located inferiorly to tremor-related higher frequency oscillations.

In macaques, motor cortex projects to the dorsal portion of the STN, and the ventral STN receives projections from prefrontal cortex. In both macaques and humans, the ventral STN has been associated with stopping movement, while the dorsal STN is more associated with motor initiation and selection. Consequently, the described tremor models find tremor-frequency oscillations (θ) originating in the STN and propagating/synchronizing to motor cortex during tremor. The described anatomical results suggest that this propagation may be specific to dorsal STN. Slowness decoding models alternatively relied upon β activity in macroelectrode recordings, perhaps reflecting anti-kinetic β bursts relayed to ventral STN from inferior frontal or supplementary motor cortex, but while prior work did not directly compare the neuroanatomical substrate of distinct PD features within or across subjects, the present disclosure directly demonstrates how alternating motor features of PD manifest along these anatomical subdivisions; surpassing the previous understanding.

The present disclosure shows that cortical PD motor decoding models were distinct from subthalamic models. In general, cortical recordings were equally capable of decoding tremor or slowness. When comparing the feature weights of these decoding models, it may be observed that opposing relationships exist in both β and γ frequency bands. Although previous studies have shown that tremor decreases β oscillations across the cortex, and others have shown increased narrowband γ activity during hyperkinetic/dyskinetic states, the present disclosure employs an otherwise unexpected “push-pull” relationship between these frequency bands in the alternating expression of tremor and slowness, and when comparing slowness and effective motor control models. While cortical β frequency oscillations are well characterized in PD, the functional role of broadband γ_{high}/hfo oscillations were less clear. Although these higher frequency oscillations overlap with phase-amplitude coupling peaks observed in cortex in unmedicated subject with PD, the described models for effective motor control states suggested that γ_{high} is specifically associated with more normal movement.

Decoding models from cortical recordings were superior to those from microelectrode recordings for all metrics. This discrepancy is likely due to differences in recording geometries. Recent studies of primate V1 found that ECoG signals contained more information than individual microelectrode LFP recordings; however, ECoG signals can be modeled as a spatial summation of microelectrodes within a grid which isolate the common local signal while canceling out random noise. In contrast, microelectrode and macroelectrode LFP recordings in the STN capture a more local signal that is disordered from PD.

Although the described cortical anatomical results (slowness-medial cortex/tremor-lateral cortex) did not reach significance, there is evidence for medial/midline cortex synchronizing with STN via β frequency oscillations The lateral aspect of the described tremor results may also align with previous functional magnetic resonant imaging (fMRI) studies demonstrating lateral somatosensory and parietal cortical interactions with cerebellar thalamus during tremor. Regardless, ECoG recordings across sensorimotor cortex provided relevant information for decoding tremor and slowness, as well as for identifying states of more effective motor function.

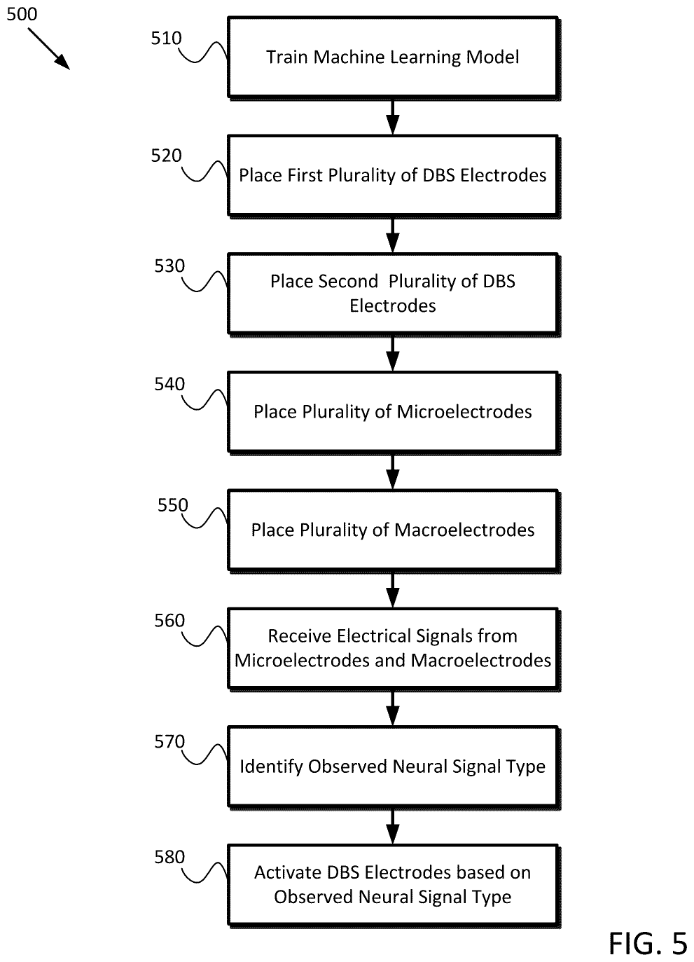

Although these data of note in isolation, the present disclosure applies the learnings for various therapeutic implications in the treatment or prophylaxis of neuromotor dysfunctions, such as PD. With advances in technology, DBS aspires towards incorporating chronic neurophysiological recordings to help guide therapeutic stimulation. While currently-applied closed-loop DBS paradigms trigger stimulation based on one or two frequency bands representing PD symptoms, the described results argue for the potential utility of a more fine-grained and targeted neurophysiological approach to PD state identification: more dorsal STN contacts may better sense signals reflecting tremor, while more ventral STN contacts may better identify signals corresponding to bradykinesia. Precise symptom-specific models could not only inform where to stimulate, but also when and how to stimulate (e.g., identifying stimulation settings to best treat tremor vs. bradykinesia). Most importantly, future neuromodulation paradigms could be derived not simply to disrupt pathological activity but actually to sustain the neurophysiological γ frequency “targets” of effective motor control. Looking ahead, chronic cortical recordings could work in concert with STN recordings to help identify precise motor states associated with specific aspects of disease expression, such as described in in greater detail with respect to method 500 described in relation to FIG. 5.

FIGS. 2A and 2B illustrate views of cursor tracing and processing for use in training a machine learning model to control therapeutically effective deep brain stimulation, according to embodiments of the present disclosure.

When gathering data to train a machine learning model 140 to differentiate different neuromodulation paradigms (e.g. signals associated with either bradykinesia or tremor), data may be gathered from various subjects who are assigned to perform various behavioral tasks to gather a training data set from which various metrics are derived.

FIGS. 2A and 2B illustrate one such behavioral task, which employs a visual-motor target tracking task to estimate motor dysfunction in a quantitative and continuous fashion. Subjects are instructed to follow a target circle that moved smoothly around the screen by manipulating a joystick or stylus with the goal of keeping the cursor within the circle. The target circle followed one of several possible paths (e.g., 210a, 210b) that were invisible to the subject, with each trial lasting 10-30 seconds. The traced path 220a, 220b are gathered from the subject's input, and are analyzed for various motor signals in coordination with brainwave data 230a, 230b simultaneously gathered from the electrodes 120 implanted in the subject while performing the tasks. Each session consisted of up to 36 trials and resulted in approximately 13 minutes of tracking data per subject per session.

Tremor amplitude 240 was calculated from band-pass (e.g., 3-10 Hz) filtered cursor traces, (TMₓ(t)=∥Cₐₓ∥). Movement speed 250 was calculated from cursor traces low-pass (e.g., at 3 Hz) filtered to remove the influence of tremor,

$$( {Speed}_{x} = ‖ \frac{{Δ}_{x}}{{Δ}_{t}} ‖ ) .$$

Although illustrated for explanatory purposes as being derived from separate performances of different tasks in FIGS. 2A and 2B respectively, the present disclosure contemplates that both tremor amplitude 240 and movement speed 2580 may be calculated for the same task. Additionally, both metrics may be averaged into non-overlapping epochs (e.g., of seven seconds) to maintain consistency with previous decoding approaches. To standardize movement speed within subjects, movement speed epochs within a session were min-max normalized into a measure of “slowness,” where 0=highest speed and 1=lowest speed.

Effective motor control was quantified as the absence of tremor and slowness measures, relative to the entire session. Each epoch's “effective motor control” measure was then calculated as

$$\frac{( 1 - Tremor ) + ( 1 - Slowness )}{2} ,$$

where values of 0 indicated symptomatic states (tremor, slowness) whereas values of 1 indicated optimal motor performance.

Tremor and slowness were compared across control and PD populations using the following linear mixed model: y_{metric}=Xₚₒₚᵤₗₐₜᵢₒₙβ+Zu+ϵ, where y_{metric} represented each epoch's metric amplitude and Xₚₒₚᵤₗₐₜᵢₒₙ represented categorical labels of populations. Linear mixed models were used to calculate the correlation between tremor and slowness across the entirety of each subject's behavioral data using the following model: yₜᵣₑₘₒᵣ=X_{slowness}β+Zu+ϵ, where yₜᵣₑₘₒᵣ represented each epoch's tremor amplitude and X_{slowness} represented the epoch's simultaneous measurement of slowness.

To determine the timescale of metric fluctuation, autocorrelograms were calculated across each PD subject's behavioral data using 100 ms epochs. The average full-width half-maximum (FWHM) of the autocorrelograms were considered the minimum time necessary to label motor metric data as a “symptomatic” period. Tremor or slowness were considered “symptomatic” if the signals exceeded the 95th percentile of aggregate control data, and sustained symptomatic periods were defined as those persisting beyond the population metric FWHM continuously. For effective motor control, epochs were labeled “symptomatic” if the signals were above the median of the PD subject's session distribution.

For effective data gathering, and therapeutic application thereafter, the positions of the electrodes 120 in the brains of the subjects was found to affect the quality of the data collected and effectiveness of treatment.

### Example 2

Experimentally, microelectrode signals were recorded using tungsten electrodes, and macroelectrode signals were recorded from circumferential contacts 3 mm above the microelectrode tips. ECoG signals were acquired using 8-contact subdural strips with 10 mm contact-to-contact spacing. All signals were acquired at 22-44 kHz and synchronized. Patients performed up to four sessions of the task, with microelectrodes positioned at different depths for each session. As microelectrodes were not independently positionable, some signals may have necessarily been acquired outside of the STN. All recorded signals were nevertheless considered and analyzed.

Neural data from the hemisphere contralateral to the subject's dominant hand were analyzed. Offline, ECoG signals were re-referenced to a common median reference within a subdural strip. All resulting signals were bandpass filtered between 2-600 Hz, and notch filtered at 60 Hz and the harmonics thereof. These resulting time series were then down sampled to 1 kHz. Time series were bandpass filtered using a Morlet wavelet convolution (e.g., wave number 7) at 1 Hz intervals, covering 3-400 Hz. The instantaneous power and phase at each frequency was then determined by the Hilbert transform. To analyze broad frequency bands, the frequencies were grouped into canonical ranges: θ/α: 3-12 Hz, β_{low}: 12-20 Hz, β_{high}: 20-30 Hz, γ_{low}: 30-60 Hz, γ_{mid}: 60-100 Hz, γ_{high} 100-200 Hz, and hfo (high frequency oscillations): 200-400 Hz. Power within the hfo band was interpreted as multiunit spiking activity, rather than discrete oscillations/ripples.

Preoperatively to inserting of the electrodes into the brains of the subjects, magnetic resonance (MR) images were obtained that included T1- and T2-weighted sequences. Pre-, intra-, and post-operative (in some cases) computed tomography (CT) scans were also acquired. Postoperative T1-weighted MR images were typically obtained 1-2 days after the operation. To reconstruct recording locations, MR and CT images were co-registered. Microelectrode depths in the brain were calculated by combining intraoperative recording depth information with electrode reconstructions obtained from postoperative images. To determine the anatomical distribution of microelectrode recording sites across subjects, preoperative T1-weighted MR images were registered to a T1-weighted MNI reference volume (MNI152 T1 2009c). The resulting subject-specific transformation was then applied to recording site coordinates, which were then assessed for proximity to the STN as delineated on the MNI PD25 atlas. ECoG contacts were segmented from intraoperative CT volumes, and were then projected onto individual cortical surface reconstructions generated from preoperative T1 volumes. Individual cortical surface reconstructions were co-registered to a standard Desikan-Destrieux surface parcellation. Contacts within sensorimotor cortex (labeled as motor or somatosensory cortex by parcellation label) were considered for the described methodology.

To investigate whether STN or cortical activity could be used to estimate co-occurring behavioral metrics, support vector regression (SVR) with a linear kernel using “scikit-learn” was applied towards multi-spectral decoding of tremor or slowness. Spectral power estimates for each canonical band (9/α, β_{low}, β_{high}, γ_{low}, γ_{mid}, γ_{high}, hfo) were further subdivided into 7 sub-bands for a total of 42 spectral features across 3-400 Hz. SVR models trained on a single electrode's spectral features were fit using 100-fold Monte Carlo cross-validation with a 2:1 train/test split of temporal epochs within a task session. Model performance was assessed by linear regression (specifically, the Pearson r-value) between the observed and predicted metric distributions. To verify that these decoded results were not spurious, a separate set of SVR models were fit with a shuffled correspondence between behavioral metric data and neurophysiological signals in the training set.

When assessing whether one type of metric (e.g. tremor) was preferentially decoded within a single type of recording (e.g. microelectrodes), SVR r-values were compared using the following linear mixed model: yᵣ₋ᵥₐₗᵤₑ=X_{metric}β+Zu+ϵ, where yᵣ₋ᵥₐₗᵤₑ represented SVR decoding r-values from a single recording and metric and X_{metric} represented categorical labels of metrics. When investigating whether one type of recording was superior at decoding a single metric, r-values were compared using the following linear mixed model: yᵣ=X_{recording}β+Zu+ϵ, where yᵣ represented SVR decoding r-values from a single recording and metric and X_{recording} represented categorical labels of recording types.

When comparing the relative ability of a model trained on tremor to decode tremor or slowness, r-values were compared within recording type using the following linear mixed model: yᵣ=X_{metric}β+Zu+ϵ, where yᵣ represented the SVR decoding r-value and X_{metric} represented the categorical labels of either the model's trained metric (tremor) or the alternate metric (slowness).

Because these SVR models used a linear kernel, the present disclosure suggests extracting SVR model coefficients (“weights”) to understand which spectral features were used to decode behavioral metrics. As linear SVR estimates of behavioral metrics (Y_{behavior}) are a combination of neural weights (Wₙₑᵤᵣₐₗ) and power estimate (Xₙₑᵤᵣₐₗ) inputs (Y_{behavior}=Wₙₑᵤᵣₐₗ·Xₙₑᵤᵣₐₗ+intercept), positive weights described the association between the presence of a specific frequency band with higher metric output values. Conversely, negative weights described the absence of a neural feature when metric output values were high.

To test whether specific clusters of features (>=3 contiguous spectral features) were consistently weighted across recordings, the distribution of each feature's SVR model weights (averaged over 3 adjacent features) across recordings were compared to the distribution of metric-shuffled SVR model weights using a contiguity-sensitive permutation test. Over 10000 iterations, each recording's SVR weight values were shuffled across the two models (empirical vs. shuffled), and the difference between individual feature distributions across electrodes was assessed using a paired t-test.

For datasets collected using the high-density STN survey, SVR models were trained using recordings throughout the STN. Specifically, data from each depth were split into 2:1 train:test sets and aggregated for whole-STN SVR model fitting. r-values from SVR models trained on high-density microelectrode data were compared to r-values from stationary microelectrode data using the following linear mixed model: yᵣ=Xₑₓₚₑᵣᵢₘₑₙₜβ+Zu+ϵ, where yᵣ represented the SVR decoding r-value and Xₑₓₚₑᵣᵢₘₑₙₜ represented the categorical labels of experiment type.

To determine if whole-STN models could decode metrics above chance, r-value distributions from empiric and metric-shuffled decoding models were compared using the Wilcoxon test. To understand if specific recording sites contained information specific to individual metrics, metrics were estimated at each depth by applying the whole-STN SVR model weights to spectral features recorded at that depth. From there, r-values for each recording/depth were calculated between estimated and observed metrics.

To compare whether specific motor features were better decoded in different regions of the STN, tremor and slowness r-values were plotted in MNI coordinate space. All voxels and their associated r-values with MER recordings were then compared with a voxel-wise paired t-test with the Analysis of Functional Neurological Images (AFNI) “3dttest++” function, available from the National Institute of Mental Health (NIMH). Each resulting voxel had an associated Z-statistic that was generated from 10000 permutations of shuffling tremor and dataset r-values across voxels using AFNI's Equitable Thresholding and Clustering (ETAC) algorithm. For cortical surface-based comparisons between tremor and slowness r-values, “3dttest++” was also used, although without the ETAC algorithm as it is not currently implemented for surface-based datasets. Thus, instead of Z-scores computed by ETAC, the T-statistic from “3dttest++” and the associated p-value thereof were examined.

Data described herein and are represented as mean±standard deviation, are presented for explanatory purposes. All statistical tests described herein, unless otherwise specified, were carried out in the “scipy” environment. P-values were adjusted for multiple comparisons wherever appropriate using the Benjamini-Hochberg procedure with q=0.05. Data points (epochs) were aggregated across trials within a single session. When comparing data aggregated across multiple subjects, linear mixed models were performed using the “statsmodels” toolbox to disentangle the effect of interest (continuous or categorical) from the random effects/unequal contributions of each subject's dataset. All linear mixed models were random intercepts models, where each random intercept corresponded to a subject's dataset, and generally followed the formula of y=Xβ+Zu+ϵ, where y represented the outcome variable, X represented the continuous or categorical predictor variables, β represented the fixed-effect regression coefficients, Z represented the subject-specific random intercepts, u represented the random-effects regression coefficients, and ϵ represented the residuals of the model fit. Once a model was fit, fixed-effect p-values were calculated from Z-scored parameter estimates (fixed-effect coefficients divided by their standard errors) against the normal distribution.

The present disclosure contemplates that different tests with different subjects may yield different data sets, which may affect the precise nature of the machine learning models, but that this variation in training data (and resulting model) is expected to still produce an improved treatment procedure according to the general spirit of the present disclosure. Similarly, although various software environments, hardware, and test procedures are discussed in the examples, the present disclosure contemplates that the skilled practitioner may make modifications to the experimental setup and the treatment setup, and that customization of the hardware and placement thereof for the treatment or prophylaxis of a neuromotor condition is expected based on the individual subject being monitored or treated, and that such modification is routine in the medical arts (e.g., does not require undue experimentation).

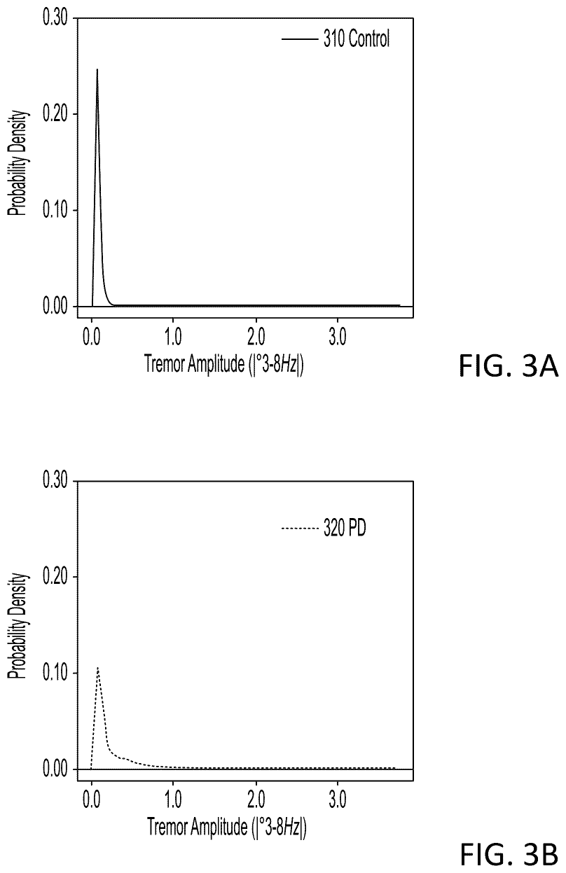

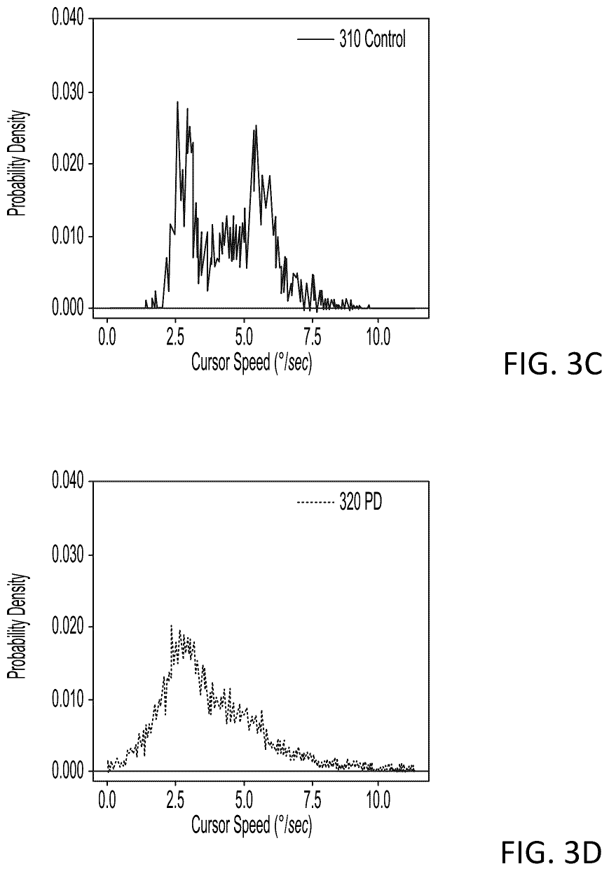

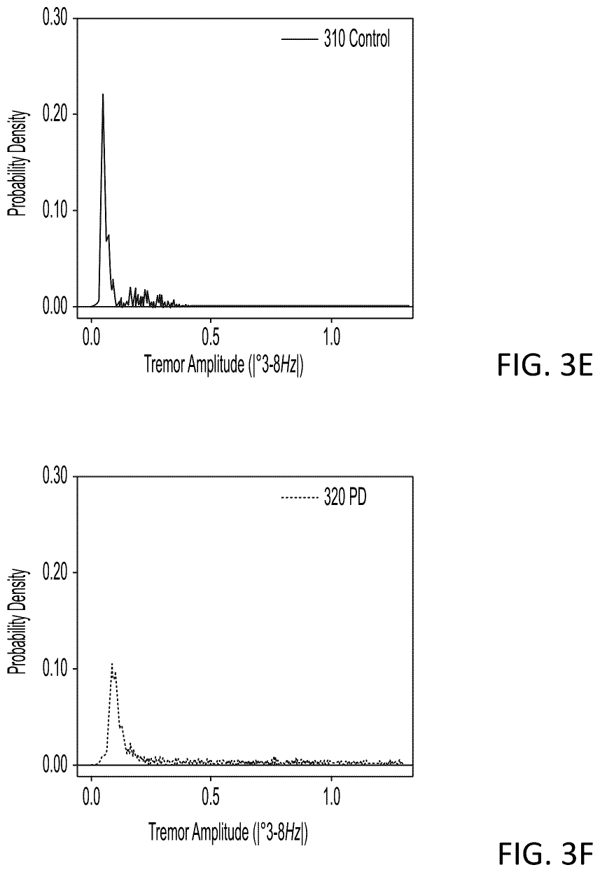

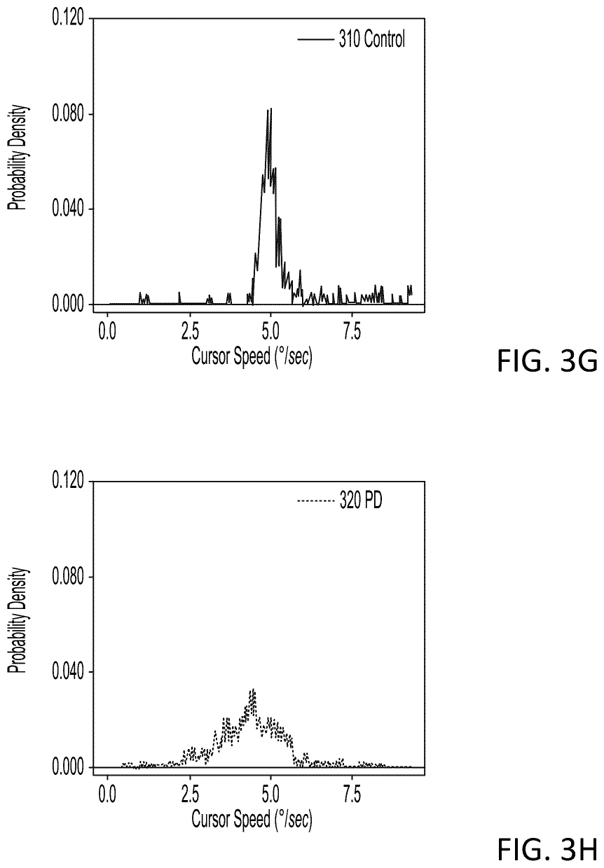

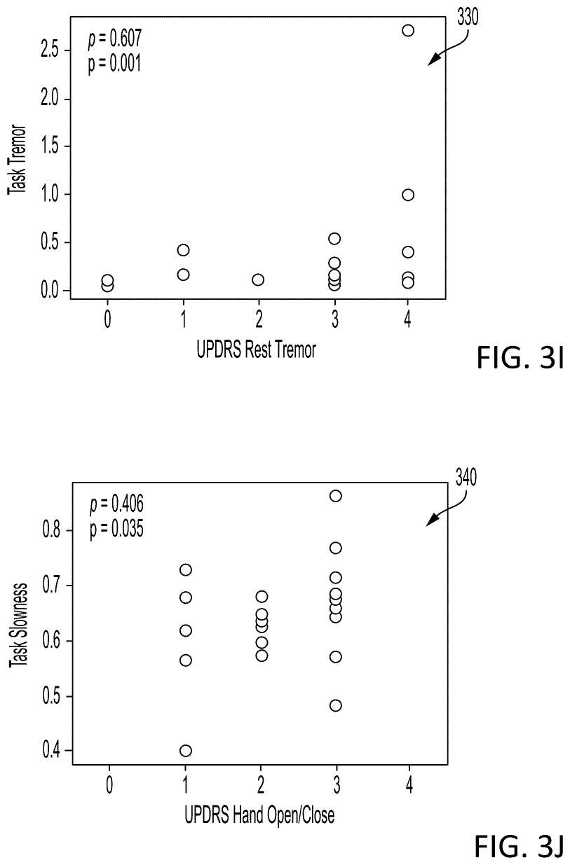

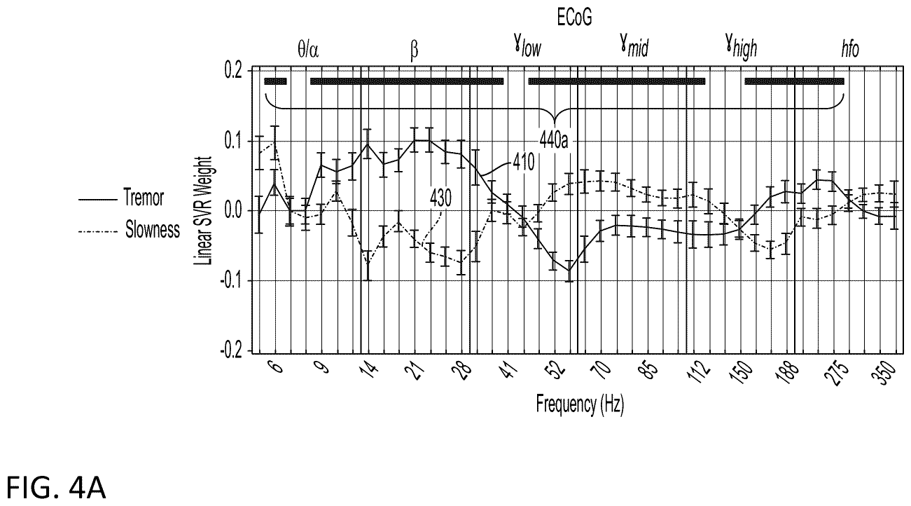

FIGS. 3A-3J illustrate plotted data from cursor tracing and processing (e.g., from the tests illustrated in FIGS. 2A and 2B) for use in performing deep brain stimulation, according to embodiments of the present disclosure.

Distributions of tremor amplitude epochs are shown in FIGS. 3A-3B and distributions of cursor speed are shown in FIGS. 3C-3D for both control subject (310) and PD subject (320) populations in a fixed-pattern task. Distributions of tremor amplitude epochs are shown in FIGS. 3E-3F and distributions of cursor speed are shown in FIG. 3G-3H for both control subject (310) and PD subject (320) populations in a random-pattern task. Data from control subjects 310 is plotted with solid lines, and data from PD subjects 320 is plotted with dashed lines.

Task-based tremor amplitudes 330 are shown in FIG. 31 and slowness amplitudes 340 are shown in FIG. 3J to corresponded to UPDRS measures of tremor or bradykinesia, where p=Spearman's correlation statistic.

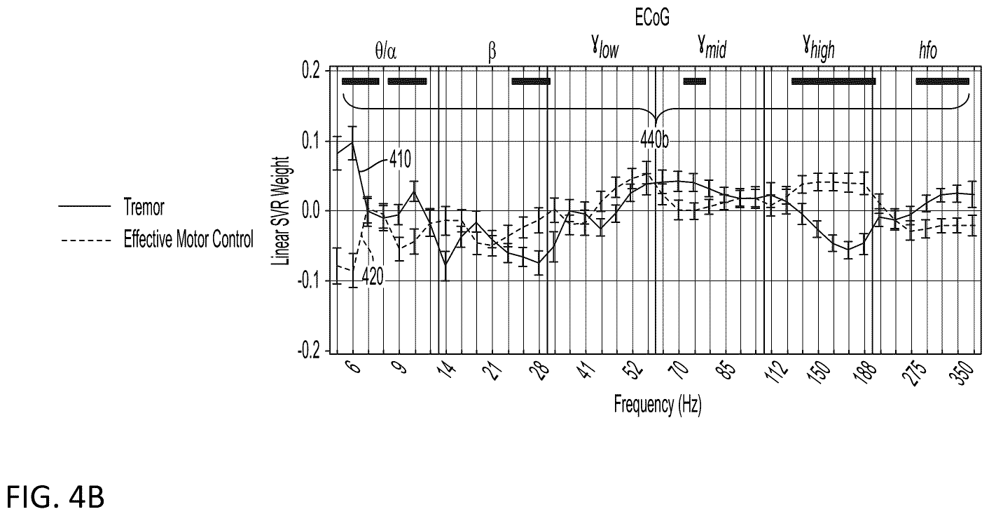

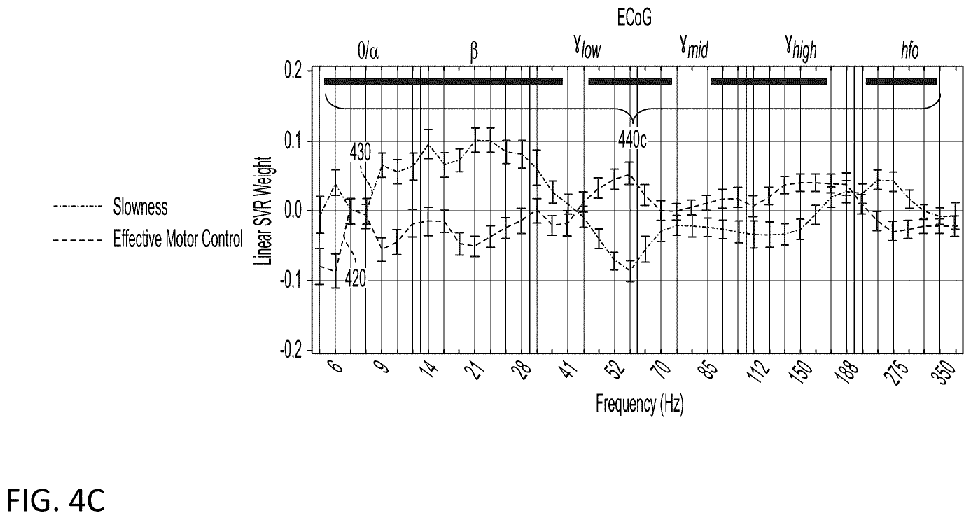

FIGS. 4A-4C illustrate plotted data for different motor effects discernable, according to embodiments of the present disclosure. In general, cortical recordings are equally capable of decoding tremor or slowness. When comparing the feature weights of these decoding models, opposing relationships are observed in both β and γ frequency bands, as is shown in FIGS. 4A-4C. As previous studies have shown that tremor 410 decreases β oscillations across the cortex, and others have shown increased narrowband γ activity during hyperkinetic/dyskinetic states, the present disclosure uses a “push-pull” relationship between these frequency bands in the alternating expression of tremor 410 and slowness 420, and when comparing slowness 420 and effective motor control 430 models.

While cortical β frequency oscillations (and their desynchronization with movement) are well characterized in PD, the functional role of broadband γ_{high}/hfo oscillations is less clear. Although these higher frequency oscillations overlap with phase-amplitude coupling peaks observed in cortex in unmedicated patients with PD, the presently described models for effective motor control states suggested that γ_{high} is specifically associated with more normal movement in line with previous research on human cortical sensorimotor mapping.

Experimentally, decoding models from cortical recordings were superior to those from microelectrode recordings for all metrics. This discrepancy is likely due to differences in recording geometries. Recent studies of primate V1 found that ECoG signals contained more information than individual microelectrode LFP recordings. However, ECoG signals can be modeled as a spatial summation of microelectrodes within a grid which isolate the common local signal while canceling out random noise. In contrast, micro- and macro-electrode LFP recordings in the STN capture a more local signal that is disordered from PD. Therefore use of unique signals from cortical, subcortical, or combinations derived from both may be used to decode distinct symptoms.

ECoG recordings across sensorimotor cortex provide relevant information for decoding tremor 410 and slowness 420, as well as for identifying states of more effective motor function 430. As plotted, the average cortical tremor and slowness decoding model coefficients for every recording along the sensorimotor cortex. The plotted lines indicate average weights, with positive/negative values reflecting a positive or negative relationship with the metric. Error bars indicate s.e.m. across subjects. Contiguous spectral features 440a-c (Black lines, top) highlight that significantly differed between tremor and slowness decoding models. The tremor signal 410 is plotted with a solid line, slowness signal 420 is plotted with a dash-dot line, and the effective motor control signal 430 is plotted with a dashed line.

FIG. 5 is a flowchart for an example method 500 of closed-loop deep brain stimulation using neural and behavioral biomarkers of specific motor features, according to embodiments of the present disclosure.

Method 500 begins at block 510, where a machine learning model is trained to differentiate first neural signals generated by a biological subject that are associated with tremor in the biological subject from second neural signals generated by the biological subject that are associated with bradykinesia in the biological subject.

In various embodiments, a corpus of signal data collected from a plurality of biological subjects may be used to train the machine learning model. In some embodiments, the training data are collected from the biological subject when performing motor tasks and are labeled by an external observer as the biological subject exhibits various symptoms of a neuromotor disorder (e.g., tremor, bradykinesia, normal motor control).

In some embodiments, the machine learning model is a Support Vector Machine (SVR), a Convolutional Neural Network (CNN), or other class of machine learning model. In various embodiments, the machine learning model is trained to differentiate the first neural signals from the second neural signals based on the first signals displaying lower frequency theta and alpha oscillations (as observed by a plurality of microelectrodes), whereas the second signals display beta oscillations and lack gamma oscillations (as observed by a plurality of macroelectrodes). In various embodiments, a machine learning model simultaneously considers multiple symptoms or effective motor function.

In various embodiments, the data gathered from subjects being treated according to method 500 may be used to update the training dataset, and the machine learning model may be re-trained or updated from time to time based on the specific neural signals of an individual subject, or an updated dataset for use with other subjects. Stated differently, the machine learning model may be a generalized model built from data from a generalized population and for use by members of the population who may not have provided training data, or a subject-specific model (which may initially be based on a generalized model) that uses data from a subject to improve the operation of the subject-specific model for use by that subject.

At block 520, an operator places a first plurality of deep brain stimulation (DBS) electrodes in a subthalamic nucleus (STN) of the biological subject.

At block 530, an operator places a second plurality of DBS electrodes in the STN of the biological subject. In various embodiments, the first plurality of electrodes (per block 520) are located within a dorsolateral region of the STN and the second plurality of electrodes are located within a ventromedial region of the STN, central relative to the first plurality of electrodes.

At block 540, an operator places a plurality of microelectrodes in the STN of the biological subject.

At block 550, an operator places a plurality of macroelectrodes in the STN of the biological subject. In various embodiments, the microelectrodes and the macroelectrodes placed in block 540 and block 550 may be preinstalled, where data gathered from these electrodes is used to train the machine learning model per block 510.

Each of the electrodes of the plurality of electrodes places per block 520, block 530, block 540, and block 550 correspond to one other electrode in the other pluralities of electrodes. For example, a first electrode of the first plurality of DBS electrodes corresponds to a first electrode of the second plurality of DBS electrodes, a first electrode of the second plurality of microelectrodes, and a first electrode of the plurality of macroelectrodes. In various embodiments, each electrode of the plurality of macroelectrodes is placed superior, relative to the biological subject, to a corresponding electrode of the plurality of microelectrodes. In various embodiments, the plurality of microelectrodes and the plurality of macroelectrodes are arranged on an anatomical trajectory for neural signals in the biological subject.

In various embodiments, the DBS electrodes, microelectrodes, and macroelectrodes placed per block 520, block 530, block 540, and block 550 may be separate electrodes, or may be different elements of the same electrodes. For example, the microelectrodes may be the tips of electrodes, whereas the macro electrodes may be a portion of the same electrodes located superior (relative to the biological subject) to the microelectrode tips. In various embodiments, the plurality of microelectrodes and the plurality of macroelectrodes are arranged on signal trajectories for neural signals in the biological subject (e.g., the pathways on which the signals travel). In various embodiments, the first plurality of DBS electrodes and the second plurality of DBS electrodes are placed in a grid-like pattern wherein the first plurality of DBS electrodes are placed within a dorsolateral region of the STN and the second plurality of electrodes are located within a ventromedial region of the STN, central relative to the first plurality of electrodes. Accordingly, the operator may select and place several individual electrodes of different types or select a combined electrode with pre-defined separation distances between various sections corresponding to the different electrode types and place a plurality of combined electrodes to place the various pluralities of different electrode types.

At block 560, the machine learning model receives electrical signals from the plurality of microelectrodes and the plurality of macroelectrodes.

At block 570, using the signals received per block 560, the machine learning model identifies a neural signal observed as one of the first neural signals (e.g., related to or indicative of tremor) or the second neural signals (e.g., related to or indicative of bradykinesia).

At block 580, the machine learning model activates a corresponding one of the first plurality of DBS electrodes or the second plurality of DBS electrodes with a therapeutically effective voltage and current to affect deep brain stimulation in the biological subject to treat a neurological disorder in the biological subject according to whether first neural signals or second neural signals were identified in block 570. Accordingly, when the neural signals are indicative of bradykinesia, the first plurality of DBS electrodes are activated with the therapeutically effective voltage and current to affect deep brain stimulation in the biological subject, while the second plurality of DBS electrodes remain inactivated (e.g., with substantially zero applied voltage and current). Similarly, when the neural signals are indicative of tremor, the second plurality of DBS electrodes are activated with the therapeutically effective voltage and current to affect deep brain stimulation in the biological subject, while the first plurality of DBS electrodes remain inactivated (e.g., with substantially zero applied voltage and current). When neither signal type is observed, the first and second pluralities of DBS electrodes remain inactivated (e.g., with substantially zero applied voltage and current). In various embodiments, the treatment may include a prophylactic treatment. In various embodiments, the treatment may be for Parkinson's Disease.

FIG. 6 illustrates a computing device 600, as may be used to provide a machine learning model 140 for the interpretation of neural signals and provision of DBS for the treatment or prophylaxis of a neuromotor disorder, according to embodiments of the present disclosure. The computing device 600 may include at least one processor 610, a memory 620, and a communication interface 630.

The processor 610 may be any processing unit capable of performing the operations and procedures described in the present disclosure. In various embodiments, the processor 610 can represent a single processor, multiple processors, a processor with multiple cores, and combinations thereof.

The memory 620 is an apparatus that may be either volatile or non-volatile memory and may include RAM, flash, cache, disk drives, and other computer readable memory storage devices. Although shown as a single entity, the memory 620 may be divided into different memory storage elements such as RAM and one or more hard disk drives. As used herein, the memory 620 is an example of a device that includes computer-readable storage media, and is not to be interpreted as transmission media or signals per se.

As shown, the memory 620 includes various instructions that are executable by the processor 610 to provide an operating system 622 to manage various features of the computing device 600 and one or more programs 624 to provide various functionalities to users of the computing device 600, which include one or more of the features and functionalities described in the present disclosure. One of ordinary skill in the relevant art will recognize that different approaches can be taken in selecting or designing a program 624 to perform the operations described herein, including choice of programming language, the operating system 622 used by the computing device 600, and the architecture of the processor 610 and memory 620. Accordingly, the person of ordinary skill in the relevant art will be able to select or design an appropriate program 624 based on the details provided in the present disclosure.

The communication interface 630 facilitates communications between the computing device 600 and other devices, which may also be computing devices as described in relation to FIG. 6. In various embodiments, the communication interface 630 includes antennas for wireless communications and various wired communication ports. The computing device 600 may also include or be in communication, via the communication interface 630, one or more input devices (e.g., a keyboard, mouse, pen, touch input device, etc.) and one or more output devices (e.g., a display, speakers, a printer, etc.).

Although not explicitly shown in FIG. 6, it should be recognized that the computing device 600 may be connected to one or more public and/or private networks via appropriate network connections via the communication interface 630. It will also be recognized that software instructions may also be loaded into the non-transitory computer readable medium 620 from an appropriate storage medium or via wired or wireless means.

Accordingly, the computing device 600 is an example of a system that includes a processor 610 and a memory 620 that includes instructions that (when executed by the processor 610) perform various embodiments of the present disclosure. Similarly, the memory 620 is an apparatus that includes instructions that when executed by a processor 610 perform various embodiments of the present disclosure.

In addition to the embodiments described above, many examples of specific combinations are within the scope of the disclosure, some of which are detailed below:

Clause 1: A method, comprising: training a machine learning model to differentiate first neural signals generated by a biological subject that are associated with tremor in the biological subject from second neural signals generated by the biological subject that are associated with bradykinesia in the biological subject; placing a first plurality of deep brain stimulation (DBS) electrodes in a subthalamic nucleus (STN) of the biological subject; placing a second plurality of DBS electrodes in the STN of the biological subject; placing a plurality of microelectrodes in the STN of the biological subject; placing a plurality of macroelectrodes in the STN of the biological subject; and in response to identifying, by the machine learning model, a given neural signal observed by the plurality of microelectrodes and the plurality of macroelectrodes as one of the first neural signals or the second neural signals, activating a corresponding one of the first plurality of DBS electrodes or the second plurality of DBS electrodes with a therapeutically effective voltage and current to affect deep brain stimulation in the biological subject to treat a neuromotor disorder in the biological subject.

Clause 2: The method of any of clauses 1 and 3-6, wherein the machine learning model is a support vector analysis model or neural network model.

Clause 3: The method of any of clauses 1-2 and 4-6, wherein the first plurality and the second plurality of DBS electrodes are implanted to cover multiple functional sub-region in the biological subject.

Clause 4: The method of any of clauses 1-3 and 5-6, wherein the plurality of microelectrodes and the plurality of macroelectrodes are arranged on an anatomical trajectory for neural signals in the biological subject.

Clause 5: The method of any of clauses 1-4 and 6, wherein the first plurality of DBS electrodes is located within a dorsolateral region of the STN and the second plurality of DBS electrodes is located within a ventromedial region of the STN, relative to the biological subject, to the first plurality of DBS electrodes.

Clause 6: The method of any of clauses 1-5, wherein the machine learning model differentiates the first neural signals from the second neural signals based on the first signals displaying lower frequency theta and alpha oscillations, whereas the second signals display beta oscillations and lack gamma oscillations.

Clause 7: A system comprising: a plurality of electrodes, including: a first plurality of deep brain stimulation (DBS) electrodes; a second plurality of DBS electrodes; a plurality of microelectrodes; and a plurality of macroelectrodes; a processor in communication with the plurality of electrodes; and a memory, storing instructions that when executed by the processor perform operations including: identifying, by a machine learning model, a given neural signal observed by the plurality of microelectrodes and the plurality of macroelectrodes as one of a first neural signal associated with tremor or a second neural signal associated with bradykinesia in a biological subject; and activating a corresponding one of the first plurality of DBS electrodes or the second plurality of DBS electrodes with a therapeutically effective voltage and current to affect deep brain stimulation in the biological subject to treat a neuromotor disorder in the biological subject associated with the given neural signal identified.

Clause 8: The system of any of clauses 7 and 9-12, wherein the machine learning model is a support vector analysis model or neural network model.

Clause 9: The system of any of clauses 7-8 and 10-12, wherein each electrode of the plurality of macroelectrodes is placed superior, relative to the biological subject, to a corresponding electrode of the plurality of microelectrodes.

Clause 10: The system of any of clauses 7-9 and 11-12, wherein the plurality of microelectrodes and the plurality of macroelectrodes are arranged on an anatomical trajectory for neural signals in the biological subject.

Clause 11: The system of any of clauses 7-10 and 12, wherein the first plurality of DBS electrodes is located within a dorsolateral region of a subthalamic nucleus (STN) and the second plurality of DBS electrodes is located within a ventromedial region of the STN, central relative to the first plurality of DBS electrodes.

Clause 12: The system of any of clauses 7-11, wherein the machine learning model differentiates the first neural signal from the second neural signal based on the first signal displaying lower frequency theta and alpha oscillations, whereas the second signal displays beta oscillations and lacks gamma oscillations.

Clause 13: A method of treatment for a neuromotor disease, comprising: placing a first plurality of deep brain stimulation (DBS) electrodes in a subthalamic nucleus (STN) of a biological subject; placing a second plurality of DBS electrodes in the STN of the biological subject; placing a plurality of microelectrodes in the STN of the biological subject; placing a plurality of macroelectrodes in the STN of the biological subject; and measuring neural signals via the plurality of microelectrodes and the plurality of macroelectrodes; in response to identifying that the neural signals are indicative of bradykinesia or tremor in the biological subject, activating one of the first plurality of DBS electrodes or the second plurality of DBS electrodes with a therapeutically effective voltage and current to affect deep brain stimulation in the biological subject.

Clause 14: The method of any of clauses 13 and 15-20, wherein the neural signals are identified as indicative of bradykinesia or tremor in the biological subject by a machine learning model trained via a data set of neuromotor tasks.

Clause 15: The method of any of clauses 13-14 and 16-20, wherein the machine learning model differentiates when the neural signal are indicative of bradykinesia from when the neural signal are indicative of bradykinesia based on whether the neural signals display lower frequency theta and alpha oscillations versus displaying beta oscillations while lacking gamma oscillations.

Clause 16: The method of any of clauses 13-15 and 17-20, wherein each electrode of the plurality of macroelectrodes is placed superior, relative to the biological subject, to a corresponding electrode of the plurality of microelectrodes.

Clause 17: The method of any of clauses 13-16 and 18-20, wherein the plurality of microelectrodes and the plurality of macroelectrodes are arranged on an anatomical trajectory for neural signals in the biological subject.

Clause 18: The method of any of clauses 13-17 and 19-20, wherein the first plurality of DBS electrodes is located within a dorsolateral region of the STN and the second plurality of DBS electrodes is located within a ventromedial region of the STN, central, relative to the biological subject, to the first plurality of DBS electrodes.

Clause 19: The method of any of clauses 13-18 and 20, wherein the neural signals are indicative of bradykinesia, the first plurality of DBS electrodes are activated with the therapeutically effective voltage and current to affect deep brain stimulation in the biological subject.

Clause 20: The method of any of clauses 13-19, wherein the neural signals are indicative of tremor, the second plurality of DBS electrodes are activated with the therapeutically effective voltage and current to affect deep brain stimulation in the biological subject.

Although the inventive concepts in the present disclosure have been described in certain specific aspects, many additional modifications and variations would be apparent to those skilled in the art. In particular, any of the various processes described herein can be performed in alternative sequences and/or in parallel (on the same or on different computing devices) in order to achieve similar results in a manner that is more appropriate to the requirements of a specific application. It is therefore to be understood that the present disclosure can be practiced otherwise than specifically described without departing from the scope and spirit of the inventive concepts described herein. Thus, embodiments of the present disclosure should be considered in all respects as illustrative and not restrictive. It will be evident to the person skilled in the art to freely combine several or all of the embodiments discussed here as deemed suitable for a specific application. Throughout the present disclosure, terms like “advantageous”, “exemplary” or “preferred” indicate elements or dimensions which are particularly suitable (but not essential) to the practice or an embodiment of the present disclosure, and may be modified wherever deemed suitable by the skilled practitioners, except where expressly required. Accordingly, the scope of the present disclosure should be determined not by the embodiments illustrated, but by the appended claims and their equivalents.

As used herein, the term “optimize” and variations thereof, is used in a sense understood by data scientists to refer to actions taken for continual improvement of a system relative to a goal. An optimized value will be understood to represent “near-best” value for a given reward framework, which may oscillate around a local maximum or a global maximum for a “best” value or set of values, which may change as the goal changes or as input conditions change. Accordingly, an optimal solution for a first goal at a given time may be suboptimal for a second goal at that time or suboptimal for the first goal at a later time.

As used herein, various chemical compounds are referred to by associated element abbreviations set by the International Union of Pure and Applied Chemistry (IUPAC), which one of ordinary skill in the relevant art will be familiar with. Similarly, various units of measure may be used herein, which are referred to by associated short forms as set by the International System of Units (SI), which one of ordinary skill in the relevant art will be familiar with.

As used herein, various terms provided with reference to the body of a biological subject are to be understood with reference to the standard anatomical position of that biological subject using anatomical terms of location (e.g., as set by the International Federation of Associations of Anatomists or the World Associate of Veterinary Anatomists) that will be understood by the person on ordinary skill in the relevant art without further explanation.

As used herein, “about,” “approximately” and “substantially” are understood to refer to numbers in a range of the referenced number, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole numbers, or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in the present disclosure, a phrase referring to “at least one of” a list of items refers to any set of those items, including sets with a single member, and every potential combination thereof. For example, when referencing “at least one of A, B, or C” or “at least one of A, B, and C”, the phrase is intended to cover the sets of: A, B, C, A-B, B-C, and A-B-C, where the sets may include one or multiple instances of a given member (e.g., A-A, A-A-A, A-A-B, A-A-B-B-C-C-C, etc.) and any ordering thereof. For avoidance of doubt, the phrase “at least one of A, B, and C” shall not be interpreted to mean “at least one of A, at least one of B, and at least one of C”.

As used in the present disclosure, the term “determining” encompasses a variety of actions that may include calculating, computing, processing, deriving, investigating, looking up (e.g., via a table, database, or other data structure), ascertaining, receiving (e.g., receiving information), accessing (e.g., accessing data in a memory), retrieving, resolving, selecting, choosing, establishing, and the like.

Without further elaboration, it is believed that one skilled in the art can use the preceding description to use the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

Within the claims, reference to an element in the singular is not intended to mean “one and only one” unless specifically stated as such, but rather as “one or more” or “at least one”. Unless specifically stated otherwise, the term “some” refers to one or more. No claim element is to be construed under the provision of 35 U.S.C. § 112(f) unless the element is expressly recited using the phrase “means for” or “step for”. All structural and functional equivalents to the elements of the various embodiments described in the present disclosure that are known or come later to be known to those of ordinary skill in the relevant art are expressly incorporated herein by reference and are intended to be encompassed by the claims. Moreover, nothing disclosed in the present disclosure is intended to be dedicated to the public regardless of whether such disclosure is explicitly recited in the claims.

What is claimed is:

1. A method, comprising:
training a machine learning model to differentiate first neural signals generated by a biological subject that are associated with tremor in the biological subject from second neural signals generated by the biological subject that are associated with bradykinesia in the biological subject;
placing a first plurality of deep brain stimulation (DBS) electrodes in a subthalamic nucleus (STN) of the biological subject;
placing a second plurality of DBS electrodes in the STN of the biological subject;
placing a plurality of microelectrodes in the STN of the biological subject;
placing a plurality of macroelectrodes in the STN of the biological subject; and
in response to identifying, by the machine learning model, a given neural signal observed by the plurality of microelectrodes and the plurality of macroelectrodes as one of the first neural signals or the second neural signals, activating a corresponding one of the first plurality of DBS electrodes or the second plurality of DBS electrodes with a therapeutically effective voltage and current to affect deep brain stimulation in the biological subject to treat a neuromotor disorder in the biological subject.

2. The method of claim 1, wherein the machine learning model is a support vector analysis model or neural network model.

3. The method of claim 1, wherein the first plurality and the second plurality of DBS electrodes are implanted to cover multiple functional sub-region in the biological subject.

4. The method of claim 1, wherein the plurality of microelectrodes and the plurality of macroelectrodes are arranged on an anatomical trajectory for neural signals in the biological subject.

5. The method of claim 1, wherein the first plurality of DBS electrodes is located within a dorsolateral region of the STN and the second plurality of DBS electrodes is located within a ventromedial region of the STN, relative to the biological subject, to the first plurality of DBS electrodes.

6. The method of claim 1, wherein the machine learning model differentiates the first neural signals from the second neural signals based on the first signals displaying lower frequency theta and alpha oscillations, whereas the second signals display beta oscillations and lack gamma oscillations.

7. A system comprising:
a plurality of electrodes, including:
a first plurality of deep brain stimulation (DBS) electrodes;
a second plurality of DBS electrodes;
a plurality of microelectrodes; and
a plurality of macroelectrodes;
a processor in communication with the plurality of electrodes; and
a memory, storing instructions that when executed by the processor perform operations including:
identifying, by a machine learning model, a given neural signal observed by the plurality of microelectrodes and the plurality of macroelectrodes as one of a first neural signal associated with tremor or a second neural signal associated with bradykinesia in a biological subject; and
activating a corresponding one of the first plurality of DBS electrodes or the second plurality of DBS electrodes with a therapeutically effective voltage and current to affect deep brain stimulation in the biological subject to treat a neuromotor disorder in the biological subject associated with the given neural signal identified.

8. The system of claim 7, wherein the machine learning model is a support vector analysis model or neural network model.

9. The system of claim 7, wherein each electrode of the plurality of macroelectrodes is placed superior, relative to the biological subject, to a corresponding electrode of the plurality of microelectrodes.

10. The system of claim 7, wherein the plurality of microelectrodes and the plurality of macroelectrodes are arranged on an anatomical trajectory for neural signals in the biological subject.

11. The system of claim 7, wherein the first plurality of DBS electrodes is located within a dorsolateral region of a subthalamic nucleus (STN) and the second plurality of DBS electrodes is located within a ventromedial region of the STN, central relative to the first plurality of DBS electrodes.

12. The system of claim 7, wherein the machine learning model differentiates the first neural signal from the second neural signal based on the first signal displaying lower frequency theta and alpha oscillations, whereas the second signal displays beta oscillations and lacks gamma oscillations.

13. A method of treatment fora neuromotor disease, comprising:
placing a first plurality of deep brain stimulation (DBS) electrodes in a subthalamic nucleus (STN) of a biological subject;
placing a second plurality of DBS electrodes in the STN of the biological subject;
placing a plurality of microelectrodes in the STN of the biological subject;
placing a plurality of macroelectrodes in the STN of the biological subject; and
measuring neural signals via the plurality of microelectrodes and the plurality of macroelectrodes;
in response to identifying that the neural signals are indicative of bradykinesia or tremor in the biological subject, activating one of the first plurality of DBS electrodes or the second plurality of DBS electrodes with a therapeutically effective voltage and current to affect deep brain stimulation in the biological subject.

14. The method of treatment of claim 13, wherein the neural signals are identified as indicative of bradykinesia or tremor in the biological subject by a machine learning model trained via a data set of neuromotor tasks.

15. The method of treatment of claim 14, wherein the machine learning model differentiates when the neural signal are indicative of bradykinesia from when the neural signal are indicative of bradykinesia based on whether the neural signals display lower frequency theta and alpha oscillations versus displaying beta oscillations while lacking gamma oscillations.

16. The method of treatment of claim 13, wherein each electrode of the plurality of macroelectrodes is placed superior, relative to the biological subject, to a corresponding electrode of the plurality of microelectrodes.

17. The method of treatment of claim 13, wherein the plurality of microelectrodes and the plurality of macroelectrodes are arranged on an anatomical trajectory for neural signals in the biological subject.

18. The method of treatment of claim 13, wherein the first plurality of DBS electrodes is located within a dorsolateral region of the STN and the second plurality of DBS electrodes is located within a ventromedial region of the STN, central, relative to the biological subject, to the first plurality of DBS electrodes.

19. The method of treatment of claim 13, wherein the neural signals are indicative of bradykinesia, the first plurality of DBS electrodes are activated with the therapeutically effective voltage and current to affect deep brain stimulation in the biological subject.

20. The method of treatment of claim 19, wherein the neural signals are indicative of tremor, the second plurality of DBS electrodes are activated with the therapeutically effective voltage and current to affect deep brain stimulation in the biological subject.
